(19) United States
(12) Patent Application Publication
(10) Pub. No.: US 2015/0323514 A1
(43) Pub. Date: Nov. 12, 2015

(54) SYSTEMS AND METHODS FOR FORECASTING BACTERIAL WATER QUALITY

(71) Applicant: UNIVERSITY OF MASSACHUSETTS, Boston, MA (US)

(72) Inventors: Robert F. Chen, Milton, MA (US); Francesco Peri, West Roxbury, MA (US); Benjamen Wetherill, Chestnut Hill, MA (US)

(21) Appl. No.: 14/698,082

(22) Filed: Apr. 28, 2015

Related U.S. Application Data
(60) Provisional application No. 61/985,229, filed on Apr. 28, 2014.

(51) Int. Cl. G01N 33/18

(52) U.S. Cl. CPC G01N 33/18

(57) ABSTRACT

Real-time, localized data may be used in a predictive model to more accurately forecast bacterial water quality.

### CROSS-REFERENCE TO RELATED APPLICATION

This application claims the benefit of priority to U.S. Provisional Patent Application Ser. No. 61/985,229 filed on Apr. 28, 2014, the entire disclosure of which is hereby incorporated herein by reference in its entirety for all purposes.

### TECHNICAL FIELD

Aspects relate to forecasting water quality and, more specifically, to systems and methods for forecasting bacterial water quality.

### BACKGROUND

Water quality for recreational purposes may be primarily determined based on the concentration of fecal indicator bacteria present in the water. Municipalities must advise the public when it is safe to recreate in water, and thus the accurate assessment of bacteria levels therein is important.

### SUMMARY

In accordance with one or more aspects, an autonomous environmental data collection station is configured to provide real-time localized data associated with a body of water. The station may comprise a buoyant base, a plurality of sensors affixed to the base and suspended at a constant depth in the body of water, a data logger in communication with the plurality of sensors, the data logger having cellular telemetry capability, at least one battery constructed and arranged to power the plurality of sensors and the data logger, and at least one solar panel constructed and arranged to charge the at least one battery.

In accordance with one or more aspects, a water quality monitoring system may comprise an autonomous environmental data collection station in communication with a body of water and configured to provide real-time localized data on at least one parameter associated with the body of water, a processor configured to receive the real-time localized data collected by the data collection station, manipulate the real-time localized data based on a predictive water quality model to determine a predictive bacteria level of the body of water, compare the predictive bacteria level to a threshold value, and output a safety recommendation based on the comparison, and a display in communication with the processor and configured to display the safety recommendation, wherein the system has an operative sensitivity of at least about 80% or a specificity of at least about 85%.

In accordance with one or more aspects, a method of generating a model to predict bacteria levels in a body of water may comprise deploying or disposing an autonomous environmental data collection station at the body of water, the autonomous environmental data collection station comprising a plurality of sensors configured to provide real-time data on a plurality of environmental parameters related to the body of water, exporting the real-time data from the plurality of sensors to an offsite database in regular intervals, simultaneously collecting water samples from the body of water and measuring the bacteria level in the body of water, sourcing data on additional environmental parameters from non-localized data collection stations, analyzing each environmental parameter to determine its predictiveness of bacteria level, selecting a plurality of analyzed environmental parameters based on their predictiveness of bacteria level, and using the selected environmental parameters to derive a predictive water quality model.

Still other aspects, embodiments, and advantages of these exemplary aspects and embodiments are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE FIGURES

Various aspects of at least one embodiment are discussed below with reference to the accompanying figures. The figures are provided for the purposes of illustration and explanation and are not intended as a definition of the limits of the invention. In the figures:

FIG. 1 presents a schematic of an environmental data monitoring station in accordance with one or more embodiments;

FIGS. 2-6B present data referenced in the detailed description and accompanying examples;

FIG. 7 presents a schematic of a floating environmental data monitoring station in accordance with one or more embodiments; and

FIGS. 8-13B present data referenced in the detailed description and accompanying examples.

### DETAILED DESCRIPTION

In accordance with one or more embodiments, a predictive water quality monitoring system may be used to better forecast bacteria levels in a body of water. The predictive water quality system may use localized, real-time data collected from an environmental data monitoring station deployed in communication with the body of water. The predictive water quality system and methods of predicting water quality disclosed herein may provide better forecasting of bacteria levels than existing predictive models and may be more effective at determining real-time and predictive recreational safety than direct sampling, and subsequent testing of the water and reporting of past bacteria levels. Bacterial water quality, and its comparison to federal or other standards, can be determined and published to inform the public about water quality and recreational health risks.

Water quality can be defined in many ways, depending on the intended use of the water and the analytical instrumentation available to take measurements. For swimming and water recreation, the primary focus of water quality analysis is the measure of fecal contamination, such as from sewage. Human sewage contains human infectious diseases, and it has been established based on a wealth of evidence that the more sewage contamination there is in a water body, the more common are instances of gastrointestinal and respiratory infections among those recreating in or around the water. The United States Environmental Protection Agency (EPA) provides guidelines concerning the safety of recreational contact with river and beach water based on concentrations of the fecal indicator bacteria Escherichia coli and Enterococcus. For example, the EPA limits E. Coli concentrations to 235 cfu/100 ml in freshwater bodies. In addition, it limits Enterococcus concentrations to 61 cfu/100 ml in freshwater bodies and to 104 cfu/100 ml in saltwater bodies. These are bacteria that are found in the intestines of warm-blooded animals, including humans. E. coli and Enterococcus are used as indicators of water quality because studies show that they have the strongest correlation to swimming-related gastroenteritis out of all studied organisms. Either indicator can be used in fresh water, but only Enterococcus is used in salt water because E. coli dies off too quickly in saline conditions.

Fecal indicator bacteria (FIB) and the pathogens they proxy originate in human intestines, and they are primarily transferred to the environment through human waste. Therefore, the primary pathway into water bodies is through raw sewage or sewers that are flushed directly into rivers or the ocean. The FIB in a given body of water can vary greatly from the FIB in a proximate body of water, and the FIB in a single body of water can fluctuate dramatically across even short time intervals. Beach and park managers are generally tasked with monitoring water quality using FIB in order to inform the public where and when it is safe to swim.

The most commonly used approach to assessing water quality is by direct sampling and analysis. FIB cannot, however, be determined from a sample in real-time. A sample of the water must be collected and taken to a laboratory for analysis. Analysis generally requires at least 24 hours to allow for bacteria cultures present in the water to grow. As a result, monitoring quality based on the analyses of water samples suffers from a severe time lag. Numerous studies have shown that bacteria levels at a given location change dramatically on a time frame of hours, and thus samples taken from a body of water 24 hours before quality is determined often do not correlate to the real-time water quality.

Some municipalities and agencies have thus attempted to forecast water-quality based on existing environmental conditions to avoid having to collect samples and wait for analysis. For example, one municipality has used wave height, wind direction and strength, and sunshine to forecast water-quality. However, these forecasts have previously been based on pre-existing data available from other, non-local sources.

Environmental factors such as rainfall, sunlight, wind, tide, and numerous other parameters may be correlated to bacteria levels in water bodies. The scientific mechanism behind these correlations is not always known, but correlations may be consistent across numerous locations and thus indicate some predictive value. For example, it has been shown that wet weather bacteria levels are statistically higher than dry weather levels in water bodies fed by storm drains, suggesting that rainfall may flush bacteria through storm drains and into the water. Some recreational areas may, for example, close after rainfall based solely on the possibility that an unsafe level of bacteria has been flushed into the water. Predictions may also be based, in part, on the bacteria levels present during the previous day. This approach persists even though many studies have concluded that rainfall by itself is only a weak indicator of bacteria levels.

Other predictive models may take into account additional environmental factors, however, these models also suffer from an undesirable time delay and the data input into the model may not be consistent with the conditions at the site of the body of water. For example, existing predictive models generally do not use localized, real-time data and instead rely on data that is provided by other services, such as nearby weather stations and geological survey sites. This data is also often collected some distance away from the body of water of interest and thus may not be representative of actual conditions. Furthermore, certain variables may not be measured by existing services, and thus current predictive models may only rely on the hydrologic and meteorological parameters measured and published by others.

In accordance with one or more embodiments, data monitoring stations are provided that collect and broadcast localized environmental, meteorological and hydrologic variables in real-time. The environmental data monitoring stations may be constructed to measure specific variables of interest and may be deployed at or in a body of water to be monitored. The environmental data monitoring stations may be networked together with other existing data sources to provide a suite of potential explanatory variables. The suite of variables may be used to generate a predictive water quality model as discussed herein.

In accordance with various embodiments, an environmental data monitoring station may be disposed on land and proximate the body of water of interest or deployed directly in the body of water. The data monitoring station may be associated with a plurality of sensors for measuring multiple environmental variables. The station may, for example, have sensors capable of measuring wind direction, wind speed, photosynthetically active radiation (PAR), air temperature, humidity, water temperature and barometric pressure. The sensors may be mounted directly on the station, tethered thereto, or otherwise be in communication with the station to provide localized, real-time data.

In accordance with some embodiments, an environmental data monitoring station may be disposed or deployed in the body of water. The data monitoring station may include a buoyant base, such as a buoy, and a plurality of sensors. The buoy may be constructed and arranged to maintain the station and/or at least one associated sensor at a constant depth in the body of water. For example, the sensor may be kept at a constant depth of between about 1 foot and about 3 feet below the surface. This may facilitate more accurate sensing. The floating data monitoring system may have, for example, a wind speed anemometer and sensors capable of measuring PAR, water temperature, barometric pressure, salinity, conductivity, wind direction, and turbidity.

The environmental data monitoring stations may be powered by any power source, and may, for example, be powered by conventional batteries, or may, in some embodiments, be solar powered.

The environmental data monitoring stations may generally be autonomous. The stations may comprise a data logger in communication with the plurality of sensors and arranged to log measurements provided by the sensors. The data logger may be configured to log data in accordance with certain time intervals, for example, every 12 hours, every six hours, every two hours, every hour, every 30 minutes, or most preferably, every 10 minutes. The data logger may have cellular telemetry capability and may send data to an offsite server in accordance with certain time intervals. These intervals may correspond to the intervals at which data is collected, or data may be sent on a different schedule. For example, data may be collected every 10 minutes and sent to the server every hour, every two hours, or at even longer intervals.

In accordance with various embodiments, the real-time data exported to a server from the environmental data monitoring station may be downloaded to a database. The database may also automatically collect and aggregate data from additional sources. For example, data may be downloaded from nearby meteorological and hydrologic stations that publish their data in accessible, web-based, formats.

In accordance with one or more embodiments, real-time data collected from a specific body of water may be used to generate a model for predicting bacteria levels within that body of water. Once the model is generated, it can then be used to assess water quality based on real-time data. The method of generating the model may comprise aggregating real-time data collected from an environmental data monitoring system discussed herein and performing statistical analysis on each variable. Water samples may be collected over a time interval, for example, one month, two months, six months, one year, two years, or more, and a laboratory analysis of relevant bacteria may be performed. The actual bacteria levels may then be used to determine the correlation coefficient of each variable with bacteria level.

In accordance with various non-limiting embodiments, each variable in the predictive model may be constructed directly from sensor data, or may for example, involve indirect manipulation of the data, such as the number of days since the last rain, modeled wind direction, or predicted tidal height, to translate it into usable form. Each environmental variable may be evaluated individually for its strength as an explanatory variable of bacteria counts. For each variable, the correlation with bacteria may be evaluated with statistical correlation coefficients, and its strength as an explanatory variable may be measured with linear regression using the coefficient of determination (R-squared) and the regression analysis of variable p-value. Each variable may, for example, be evaluated for a range of time frames. For example, the variable may be evaluated at time of sample, 24 hours prior to sample, 48 hours prior to sample, 72 hours prior to sample, 25-28 hours prior to sample, 49-72 hours prior to sample, 73-96 hours prior to sample, and at additional time frames deemed appropriate. Variables may be selected for further analysis based on p-value, for example, if the variable has a p value (<0.05), a high R² coefficient, and high correlation.

From this initial analysis, a subset of strong explanatory variables may be identified. The identified variables may then be used to develop predictive models. The model may be any one of, or a combination of, a multiple linear regression model, a logistical regression model, or an algorithmic model. For example, a non-limiting multiple linear regression model may be constructed following ordinary least squares regression and have the form:

(Y|X)=β₀+β₁X₁+β₂X₂+ . . . +βₙXₙ+ε

Where:
C(Y|X) represents a function of Y on X
Y represents the log of bacteria counts (the dependent variable)
Xₙ represents an environmental condition (an explanatory variable)
βₙ represents the regression coefficient for each explanatory variable
ε represents the error term.

For logistic regression, the function C(Y|X) may be replaced by the logic function, which converts a linear response to a binary probability between 0 and 1. The logistic regression models may be constructed in the following form, following maximum likelihood regression:

ln(p/1−p)=β₀+β₁X₁+β₂X₂+ . . . +βₙXₙ+ε

Where:
p represents the probability of exceeding a threshold.

An initial model may be generated with all variables, and then the model may be improved by selectively removing variables. For linear regression, strength of fit of the model may be evaluated by minimizing the model p-value (regression analysis of variance) and maximizing the model R² (coefficient of determination). Variables may, for example, be removed one at a time based on individual coefficient p-values (two-sided t-tests), and the regression may be rerun between each variable change. Interaction terms may be added and evaluated in cases where interaction was logical. In accordance with some non-limiting embodiments, a predictive model may have no more than five explanatory variables. For example, a predictive model may have between about 2 and about 5 variables.

The final models may be evaluated for model accuracy, sensitivity (true-positive rate), and specificity (true-negative rate). In accordance with some non-limiting embodiments, a final model may have a sensitivity of at least about 80% and/or a specificity of at least about 85%.

In accordance with one or more embodiments, a water quality monitoring system may include an autonomous environmental data collection system configured to provide real-time localized data on parameters associated with a specific body of water, and a processor configured to receive the real-time localized data. The processor may manipulate the real-time data based on a predictive water quality model to determine a predictive bacteria level of the body of water. The processor may also utilize additional data to determine the predictive bacteria level. The model may predict E. coli bacteria levels in the body of water, or may predict Enterococcus bacteria levels in the body of water.

The processor may then compare the predictive bacteria level to a threshold value, for example, established federally by the EPA, and output a safety recommendation based on the comparison. A display may be in communication with the processor. The display may alert the public to the safety recommendation. For example, the logistic model will display whether a body of water is safe or not safe for recreational activities. The linear model may provide users with a specific predicted bacterial level. The display may, for example, be an online website, a mobile application, or some other virtual presentation of the recommendation, or may, for example, be a sign or flag manually posted at the site of the body of water. A municipality or other governing body may provide more accurate safety recommendations based on real-time localized data as discussed herein and/or may take other action in connection with the safety of the body of water such as the treatment thereof.

In accordance with one or more embodiments, a predictive water quality monitoring system may provide cost savings over manually sampling water to determine bacteria levels. Each time a body of water is sampled, such as daily, a technician must physically go to the water, take a sample, and return it to the laboratory. In addition to compensating the technician for his time, there is an additional cost associated with the laboratory testing. For waters that are sampled regularly, these costs can become quite large. A predictive water quality monitoring system may reduce costs by reducing sampling and thereby reducing associated laboratory costs. In some embodiments, sampling can instead be performed every other day, every few days, weekly, monthly or less frequently so as to simply confirm the accuracy of the predictive model. The accuracy may be compromised, for example, upon a shift in land use, climate changes, hydrological changes such as a dam, or water treatment systems.

In accordance with one or more embodiments, the systems may be self-updating. After an initial predictive model is established, real-time localized data that is collected for input to the model for predictive purposes may also be used to update the model. The model may be updated periodically or continuously, manually or automatically. In at least some embodiments, the model may be updated daily. By automating the regression analysis, software can be written that can periodically recalculate coefficients to update the model to the most recent data.

In accordance with one or more embodiments, high-frequency data may enable the use of average conditions instead of a single data point. High-frequency real-time data may also allow predictive models to be updated more frequently, such as more than once per day. For example, the predictive models may be updated hourly. This may beneficially provide information about changes to water quality throughout the day that daily sampling could never provide. For example, the prediction can be made every hour or two with predictions that can change with time of day rather than one prediction for the whole day and therefore users of the data can plan their recreational uses of the water body accordingly just as they use weather predictions to determine what to wear and when to go out.

The function and advantages of these and other embodiments will be more fully understood from the following examples. The examples are intended to be illustrative in nature and are not to be considered as limiting the scope of the systems and methods discussed herein.

### Example 1

A river was selected as a site for generating a predictive model in accordance with one or more embodiments, and the model was tested for its accuracy, sensitivity, and selectivity. The river selected was a water body flowing in a single direction, not open to swimming but used for boating and other water activities. The river was a fresh water body and thus the primary water quality metric of interest was E. coli concentrations.

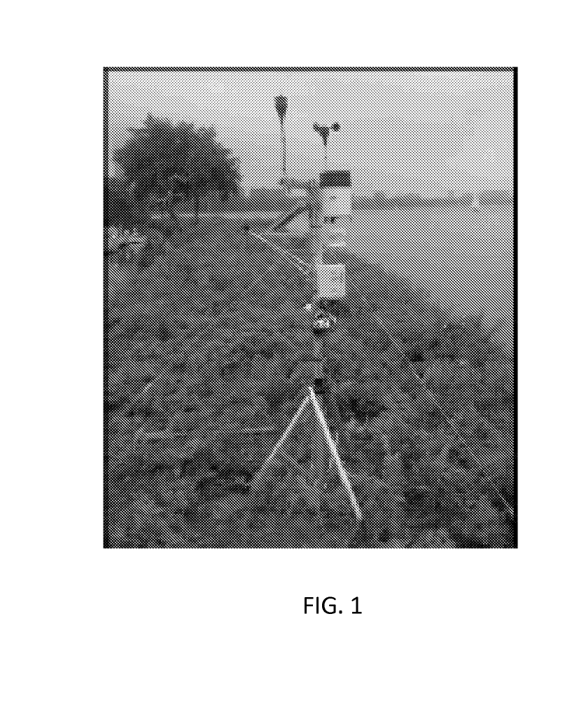

An autonomous environmental data collection system, shown in FIG. 1, was installed on an island in the river basin. The station included a data logger with cellular telemetry capability (Onset U30 GSM), a wind direction sensor (Onset S-WDA-M003), a wind speed anemometer (Onset S-WSA-M003), a sensor for photosynthetically active radiation (PAR) (Onset S-LIA-M003), an air temperature and humidity sensor (Onset S-THB-M002), a water temperature sensor (Onset S-TMB-M017), a barometric pressure sensor (Onset S—BPB-CM50), and a rain gauge (Onset S-RGA-M002). The station was powered by a 4.5 V battery, which was kept charged by a 6 W solar panel.

Initially, the water temperature sensor was located on the river bottom about 1.2 meters from shore in a water depth that varied from 0 to 60 cm in response to rain and flood events. The variations in water depth caused rapid fluctuations in temperature as the depth of the sensor changed that were not related to river temperature fluctuations. To guard against inaccurate water temperature data, a water temperature sensor was instead suspended below a buoy at a constant depth of about 20-25 cm approximately 4.5 m from shore. This position was chosen because it represents standard bacterial sampling depth, which is below the surface affected by wind and floating material, but is near the surface where most human exposure occurs.

Data was logged every 10 minutes and sent by cell-phone based telemetry to an Onset server every 60 minutes. An automatic web-based job was created to download the data to a local database every 2 hours. Another automatic job was also created to retrieve river flow data from a U.S. Geological Survey flow gage located 14 km upriver of the monitoring station. This network of data was then incorporated in a web-based model that predicted bacteria levels. The predictions were updated every hour.

Bacteria samples were collected and analyzed in a laboratory. Samples were collected two times per week for about approximately three months. Sampling was conducted within a two to three hour window in the morning to avoid variability in the measurements. Samples were collected from a small boat in the middle of the river at approximately the same location as previous sampling was performed. Samples were collected in sterile bottles at 15 cm depth and stored on ice for no more than 6 hours until analysis. Laboratory analysis of E. coli followed USEPA's Modified E. coli Method 1603. This procedure involves filtering an aliquot of sample through a sterile membrane and then placing the filter on modified mTEC agar and incubating for 2 hours at 35° C. and then 22 hours at 44.5° C. The final E. coli concentration was deduced by counting red colonies after incubation.

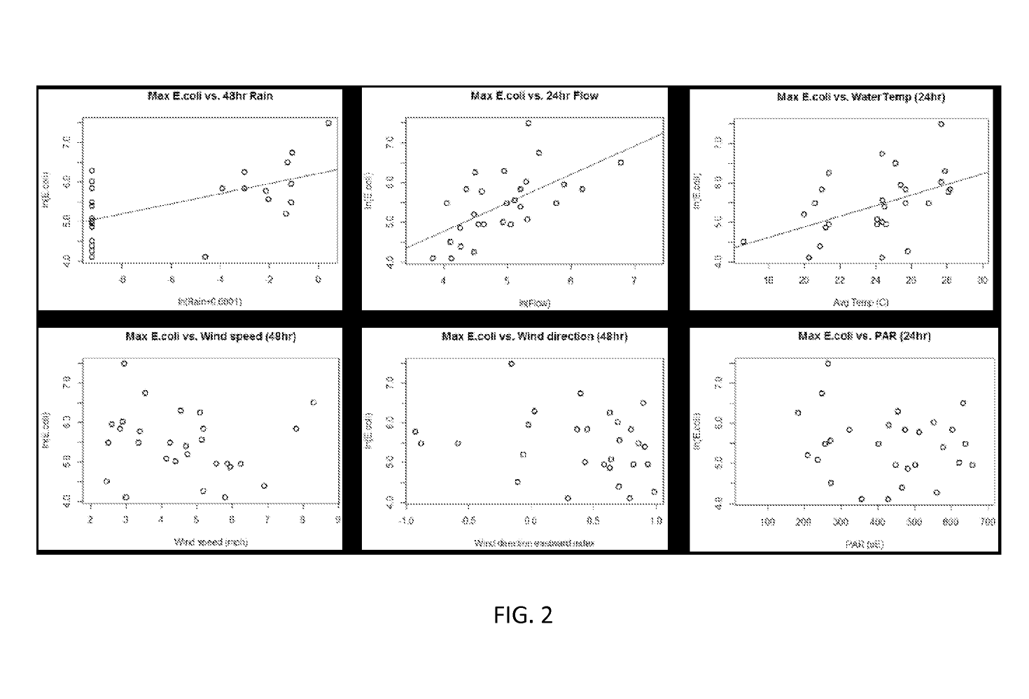

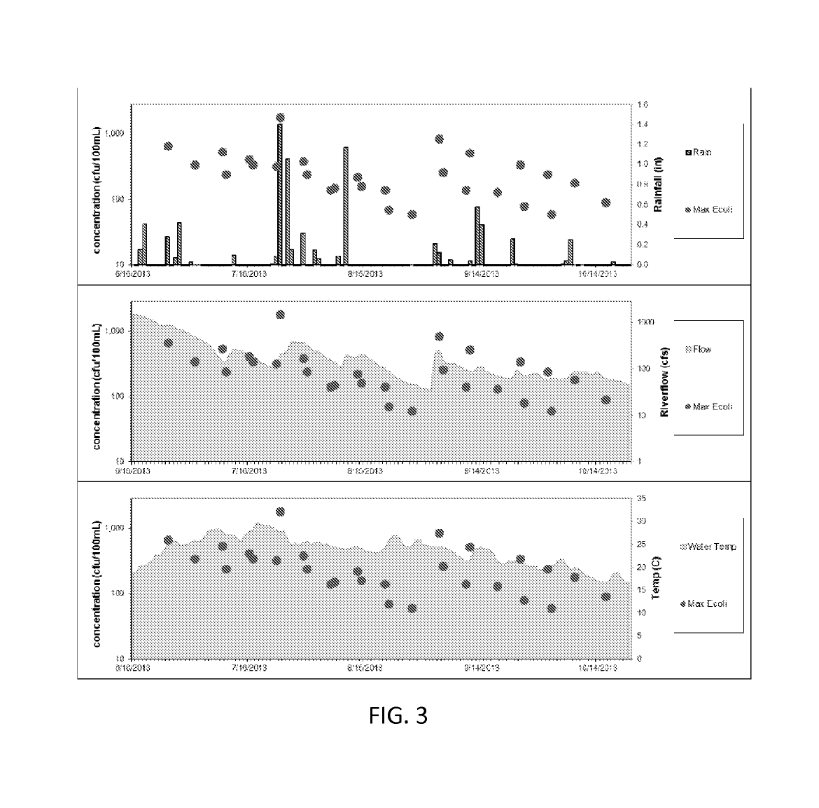

FIG. 2 shows scatterplot comparisons of environmental variables vs. river E. coli samples. Variables with strong correlation are shown in the top row, and variables with poor correlation are shown in the bottom row. As shown in FIG. 3, analysis of the strength of correlation of each of the environmental variables highlighted rainfall, river flow, and water temperatures as the strongest correlations.

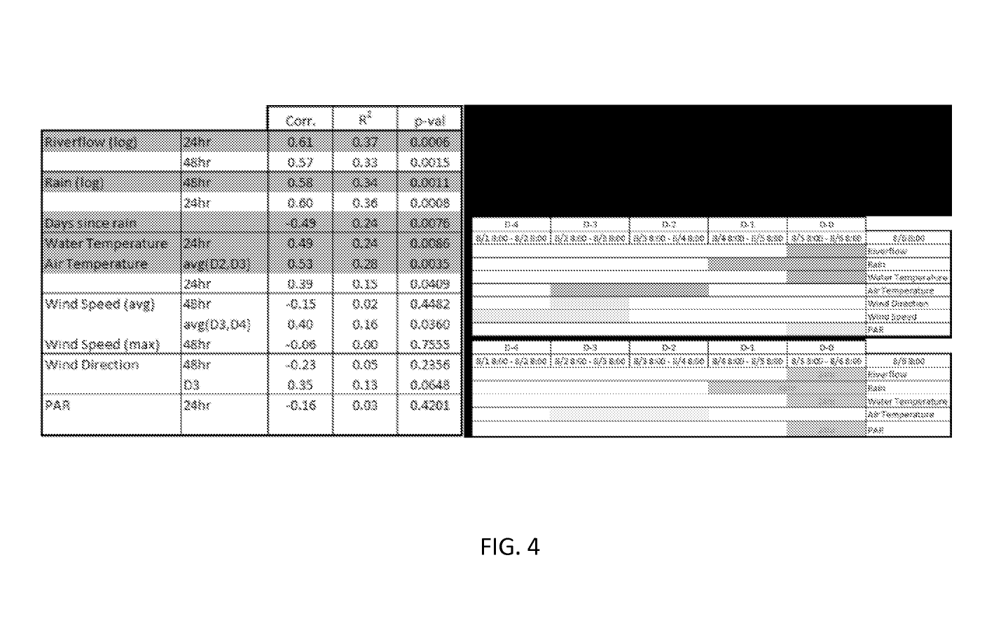

FIG. 3 shows time-series comparisons of rainfall, river flow, and water temperature versus maximum daily E. coli samples collected from the river. Correlations for these three variables are clearly visible in these time series graphs. Rain and river flow each had correlation coefficients greater than 60% with water temperature's correlation coefficient also strong at 48%, and each was a strong predictor of E. coli with simple linear regression p-values less than 0.01. FIG. 4 shows the analysis of each variable across multiple time periods. For river flow, the previous 24-hour average was a slightly stronger predictor than the 48-hour average, so the 24-hour average was used in regression modeling. FIG. 4 (left) shows a summary of statistical correlations between environmental variables and river E. coli samples. Strong correlations are highlighted in blue. Depictions of time periods with strongest correlations to E. coli are shown in the top-right, and depictions of time periods with strongest regression characteristics are shown in the bottom right. Rainfall was statistically similar to flow, but since rainfall is more sporadic than flow, it was concluded that the 48-hour time period would be more robust in other years than the 24-hour time period. The number of days since the last rain was also identified as a strong predictive variable. This predictive capacity is evident in the time-series graph shown in FIG. 3, which shows the strongest bacterial spikes after the longest dry periods. Water temperature was a strong predictor through 96 hours prior to sampling, but it was statistically strongest for the previous 24 hours. Since water temperature changes gradually, it was considered safe to use the 24-hour average in modeling.

Referring to FIG. 2, wind speed and wind direction showed little visible correlation. However, analysis of different time lags for these variables highlighted some interesting relationships, shown in FIG. 4. Both wind speed and wind direction showed fairly strong evidence of correlation for the time period 3 days prior to the bacteria sample date (regression p-values <=0.06). This may be explainable with reference to air temperature. As mentioned above, air temperature is a strong predictor of bacteria levels if time-lagged about 2 to 3 days. Additional analysis showed that air temperature is also a strong predictor of both wind speed (p-value=0.0016) and wind direction (p-value=4×10-5). This is a reasonable relationship because wind is primarily produced by variances in atmospheric air temperature. Since wind speed and wind direction had poor predictive strength in the 24 to 48-hour window and did not hold up well in model development, this study concluded that they are secondary correlates through air temperature and add little to no additional value.

Referring again to FIG. 2, PAR showed little visible correlation and demonstrated no strength as an individual predictive variable, but PAR was a strong predictive variable in multiple regression models. The reason for this incongruity is not obvious. Irradiance is expected to cause bacterial mortality, so it is logical that it would play an important role in a model as a negative factor. It is possible that the interaction effect of PAR with the other variables is significant even when the individual effect is not. Analysis of the predictive strength of PAR in simple linear regression for other variables showed that it was a strong positively correlated predictor for water temperature (p-val=7×10-6), wind speed (p-val=5×10-4), wind-direction (p-val=2×10-4), and river flow (p-val=3×10-3).

Correlations with temperature and wind are logical. Correlation with river flow might be reflecting the fact that higher flows occur on days after rain when the sky has cleared. However, the negative predictive strength of PAR in bacterial regression models could be a result of its positive correlation with both temperature and river flow which was also included in the model. Since PAR has a negative effect on bacteria, it may be necessary to include it to moderate the positive effect of temperature and flow.

Based on this analysis of individual variable correlations, the following variables were chosen for further analysis in regression models: 48-hour rain, days since rain, 24-hour water temperature, 72-hour air temperature, 96-hour air temperature, ⅔-day time-lagged air temperature, 24-hour river flow, 24-hour PAR, 3-hour PAR, 48-hour wind speed, and 48-hour wind direction. Through backward elimination, two best-fit models were developed:

Linear: ln(E)=2.34−0.068D+0.12T₂₄+0.29ln(F₂₄)−0.0021P₂₄

Logistic: ln(p/1−p)=−9.96+0.46ln(R₄₈+10−4)+0.54T₂₄

Where:
E=modeled E. coli counts (CFU/100 mL)
p=modeled probability of exceeding swimming threshold
D=days since last rain greater than 0.1″
T=average water temperature (° C.)
F=average river flow (cfs)
P=average PAR (uE)
R=total rainfall (in)
W=average wind speed (mph)
M=average wind magnitude or direction (vector)
A=average air temperature (° C.)
Subscripts represent hours included in each average.

Table 1 and Table 2 summarize the model development process with corresponding goodness of fit values for each step. In the linear model development, it was not completely clear whether to choose the model with river flow or the model without it. Ultimately the model with river flow was selected because logically it makes sense to have at least one variable reflecting rain volume, and because the model without river-flow was less flexible, having a much lower R² value when air temperature was substituted for water temperature. In logistic model development, the best-fit model actually has only two variables, rain and temperature. This is ideal, considering the fact that the sample size was only 28.

TABLE 1

| Variables | R² | F-statistic | p-value |
|---|---|---|---|
| In(R₄₈ + 10⁻⁴), D, T₂₄, A₇₂, A₉₆, A₍₂₋₃₎, In(F₂₄), P₂₄, In(R₄₈ + 10⁻⁴): D | 0.642 | 3.59 on 9 + 18 d.f. | 0.0101 |
| In(R₄₈ + 10⁻⁴), D, T₂₄, A₇₂, A₉₆, A₍₂₋₃₎, In(F₂₄), P₂₄ | 0.638 | 4.18 on 8 + 19 d.f. | 0.0050 |
| In(R₄₈ + 10⁻⁴), D, T₂₄, A₉₆, A₍₂₋₃₎, In(F₂₄), P₂₄ | 0.631 | 4.88 on 7 + 20 d.f. | 0.0024 |
| In(R₄₈ + 10⁻⁴), D, T₂₄, A₉₆, In(F₂₄), P₂₄ | 0.626 | 5.85 on 6 + 21 d.f. | 0.0010 |
| In(R₄₈ + 10⁻⁴), D, T₂₄, In(F₂₄), P₂₄ | 0.624 | 7.29 on 5 + 22 d.f. | 0.0004 |
| D, T₂₄, In(F₂₄), P₂₄ | 0.611 | 9.05 on 4 + 23 d.f. | 0.0002 |
| D, A₍₂₋₃₎, In(F₂₄), P₂₄ | 0.596 | 8.48 on 4 + 23 d.f. | 0.0002 |
| D, T₂₄, P₂₄ | 0.584 | 11.2 on 3 + 24 d.f. | 0.0001 |
| D, A₍₂₋₃₎, P₂₄ | 0.544 | 9.54 on 3 + 24 d.f. | 0.0002 |
| Also evaluated P_{3 hr}, W₄₈, M₄₈, W₄₈: M₄₈ - negative effect for all | | | |

TABLE 2

| Variables | Res. deviance | AIC |
|---|---|---|
| In(R₄₈ + 10⁻⁴), D, T₂₄, A₍₂₋₃₎, In(F₂₄), P₂₄, In(R₄₈ + 10⁻⁴): D | 20.73 on 21 d.f. | 36.7 |
| In(R₄₈ + 10⁻⁴), D, T₂₄, In(F₂₄), P₂₄, In(R₄₈ + 10⁻⁴): D | 20.73 on 21 d.f. | 34.7 |
| In(R₄₈ + 10⁻⁴), D, T₂₄, P₂₄, In(R₄₈ + 10⁻⁴): D | 20.74 on 22 d.f. | 32.7 |
| In(R₄₈ + 10⁻⁴), D, T₂₄, In(R₄₈ + 10⁻⁴): D | 21.16 on 23 d.f. | 31.2 |
| In(R₄₈ + 10⁻⁴), D, T₂₄ | 21.83 on 24 d.f. | 29.8 |
| In(R₄₈ + 10⁻⁴), T₂₄ | 22.10 on 25 d.f. | 28.1 |
| In(R₄₈ + 10⁻⁴), A₍₂₋₃₎ | 23.09 on 25 d.f. | 29.1 |
| Also evaluated A₇₂, A₉₆, P₃ₕᵣ, W₄₈, M₄₈, W₄₈: M₄₈ - negative effect for all | | |

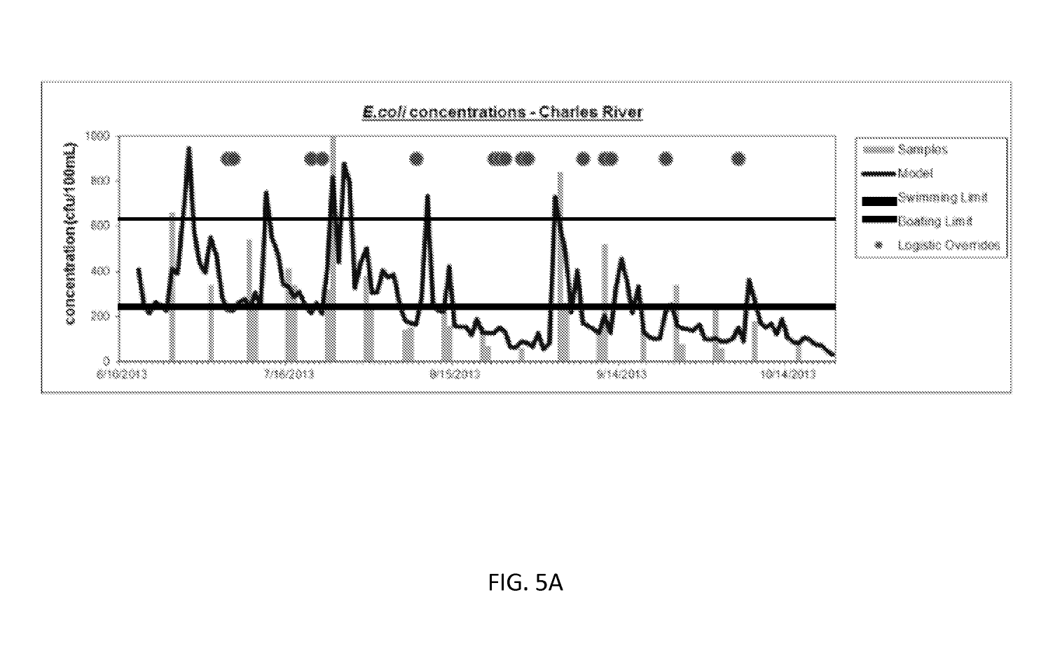

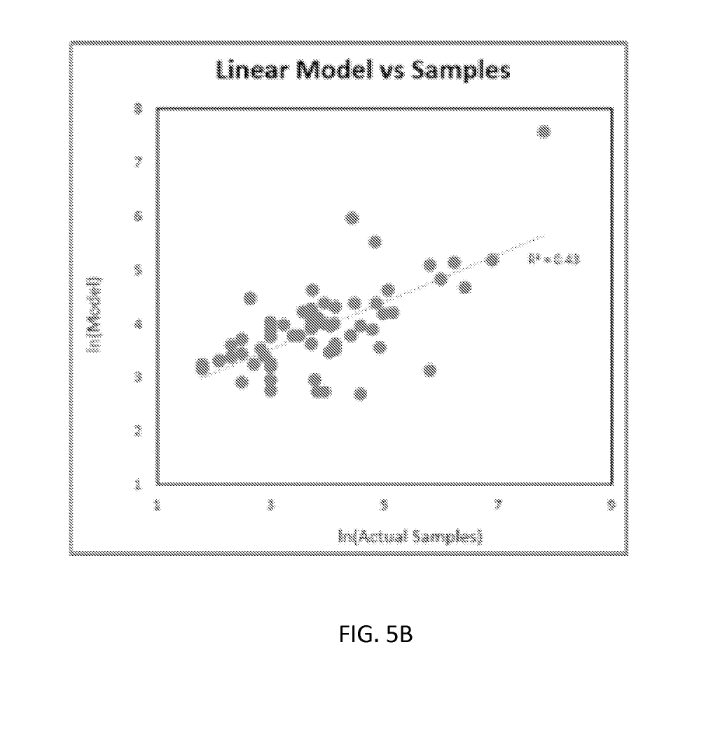

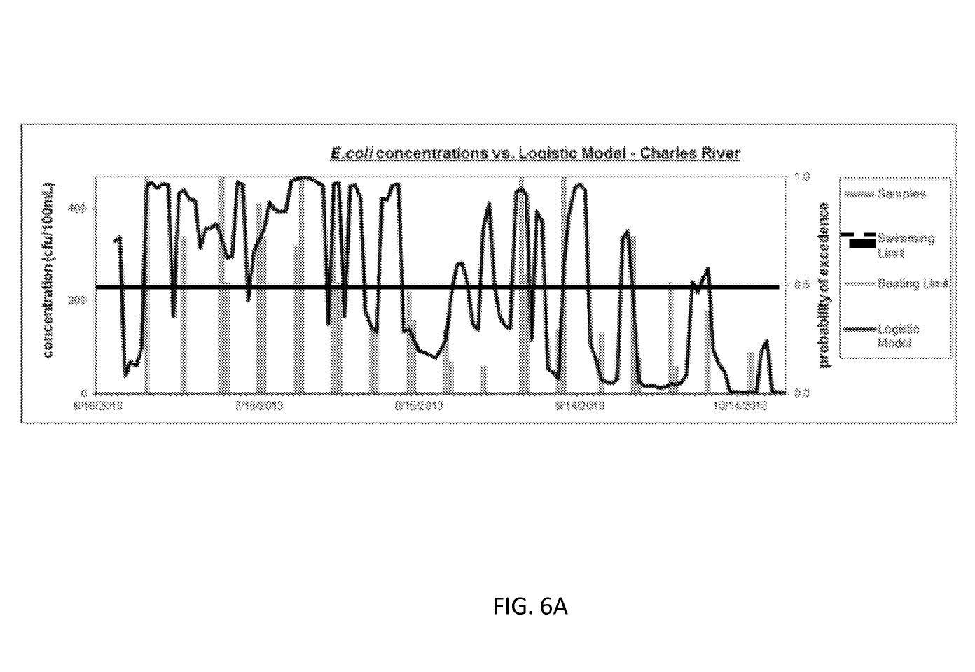

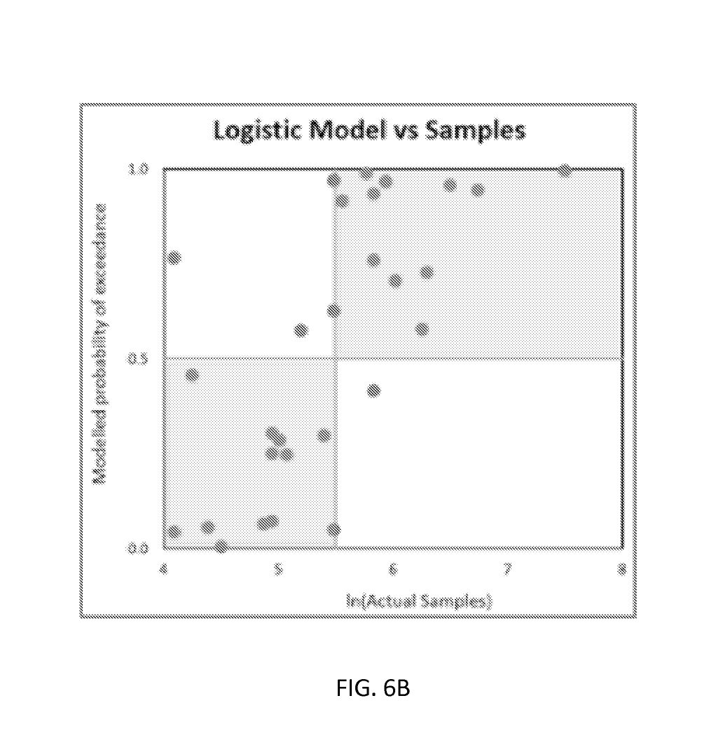

Once models were developed, it was possible to study the predictive strength of the models and the value of real-time sensors and real-time predictions. A summary of the linear model performance is shown in FIGS. 5A and 5B. The linear model had a true-positive rate (sensitivity) of 80% and a true-negative rate (specificity) of 85%. In other words, when run once per day at 8:00 am, it correctly predicted 80% of actual bacterial exceedances and 85% of actual good water quality days. A summary of the logistical model performance is shown in FIGS. 6A and 6B. The logistical model produced even better results with 87% true-positive rate and 85% true-negative rate

### Example 2

A beach was selected as a site for generating a predictive model in accordance with one or more embodiments. The model was then tested for its accuracy, sensitivity, and selectivity. The beach was open to swimming, and the water was a multidirectional open body of saltwater. The primary water quality metric of interest was Enterococcus bacteria.

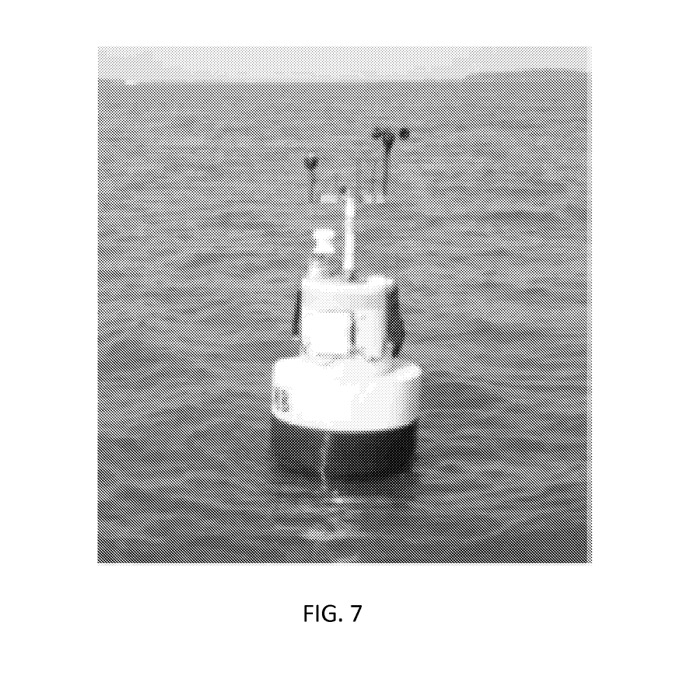

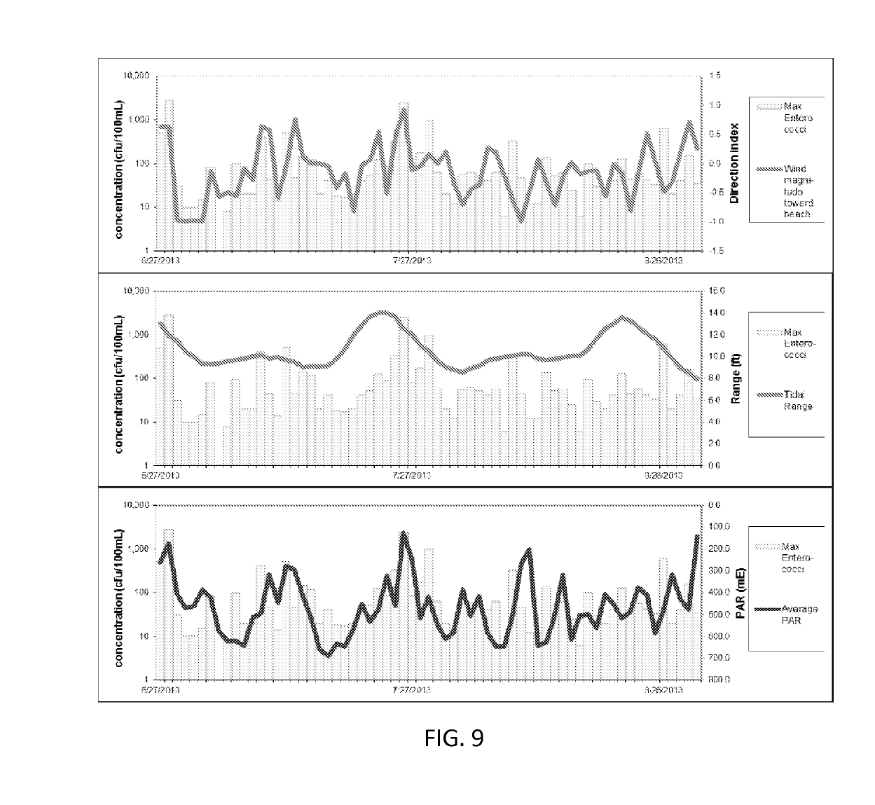

A custom buoy, shown in FIG. 7, was designed and deployed in the water about 1200 meters offshore in 2.5 meter of water, as measured at low tide. The buoy included a data logger with cellular telemetry capability (Onset U30 GSM), a wind speed anemometer (Onset S-WSA-M003), a sensor for Photosynthetically Active Radiation (PAR) (Onset S-LIA-M003), an air temperature and humidity sensor (Onset S-THB-M002), a water temperature sensor (Onset S-TMB-M017), a barometric pressure sensor (Onset S—BPB-CM50), a salinity/conductivity sensor (In-Situ Aqua TROLL 200), and a turbidity fluorometer (Turner Cyclops 7T) (FIG. 9). The station was powered by two parallel 4.5 V batteries, which were kept charged by two 3 W solar panels. Data was logged every 10 minutes and sent by cell-phone based telemetry to an Onset server every 2 hours. An automatic web-based job was created to download the data to a local database every 2 hours. Another automatic job was also created to retrieve tide data from a National Oceanic and Atmospheric Administration tide gauge. A third automatic job downloaded rain and wind direction data from an existing weather station located on the roof of an 11-story University building. It used an Onset data logger (Onset U30 Ethernet) and had a rain gauge (Onset S-RGA-M002) and a combined wind speed and direction sensor, as well as other standard meteorological sensors not used, including air temperature, relative humidity, atmospheric pressure, and PAR. This network of data was then incorporated in a web-based model that predicted bacteria levels. The predictions were updated every two hour. Table 3 provides a summary of the data preparation conducted.

TABLE 3

| Sensor output | Derived variables |
|---|---|
| Tide height | Tide height |
| | Tidal maximum—maximum height over a time period |
| | Tidal range—maximum range between low and high tides over a time period |
| | Tide phase—Boolean reflection of tide direction (ebb/flood) at a point in time |
| Water temperature | Average Water Temp—average over a time period |
| Air temperature | Average Air Temp—average over a time period |
| PAR | Average PAR—average over a time period |
| | Day phase—Boolean reflection of the light level (light/dark) at a point in time |
| Wind speed | Average Wind speed—average over a time period |
| | Maximum Wind speed—maximum over a time period |
| Salinity | Corrected Salinity—salinity corrected for bio-fouling |
| | Average Salinity—average corrected salinity over a time period |
| Turbidity | Corrected Turbidity—turbidity corrected for bio-fouling |
| | Average Turbidity—average corrected turbidity over a time period |
| Rain | Total Rain—sum of rain over a time period |
| | Days since—number of days since last significant rain |
| Wind direction | Northerly/Westerly vectors—directional components of wind direction |
| (see Appendix 1 for a detailed | Average Northerly/Westerly vectors—average vectors over a time period |
| description of logic) | Average Wind direction—direction calculated from average vectors |
| | Magnitude of wind in specific direction—directional component of average wind |
| | direction adjusted for consistency of wind direction over a time period |
| River flow | Average Flow—average over a time period |

Bacteria samples were collected daily for the duration of the swimming season, which comprised about two months. Consistent timing of samples is preferred due to tidal variability, and high tide is more typical of ocean waters rather than waters subjected to land/stream influences. For this reason, samples were collected within about three hours of high tide in 200 mL sterile bottles at 0.3 m depth in 1 m of water and stored on ice packs until analysis for no more than 6 hours. Laboratory analysis of Enterococcus followed USEPA Method 1600. This procedure involves filtering an aliquot of sample through a sterile membrane and then placing the filter on mEI agar and incubating for 24 hours at 35° C. The final Enterococcus concentration was deduced by counting blue colonies after incubation.

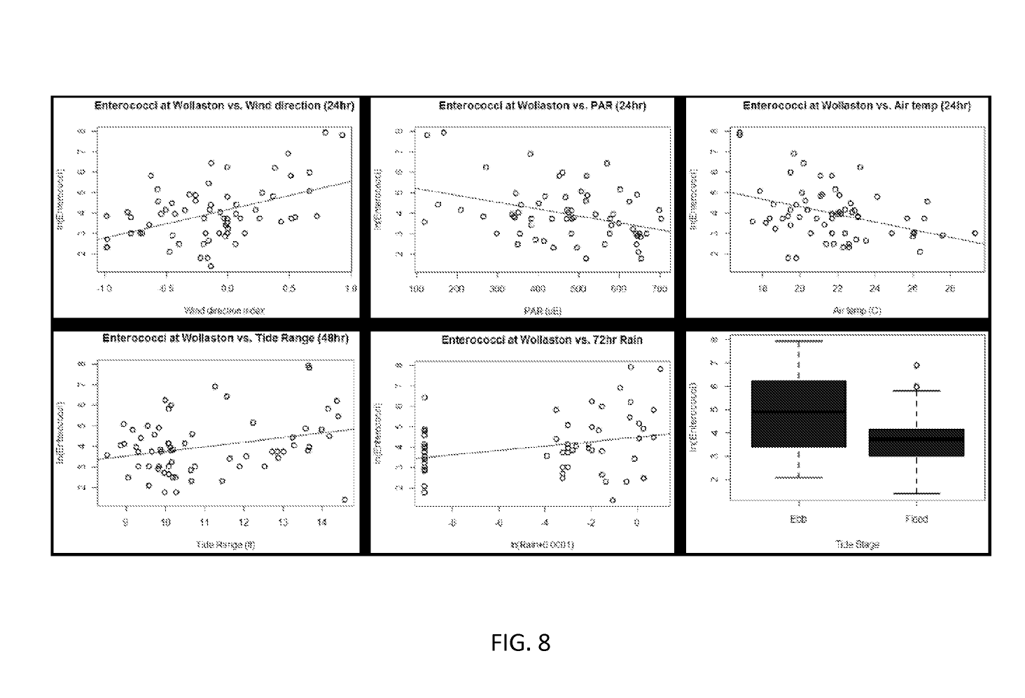

FIG. 8 shows scatterplot comparisons of environmental variables verses Enterococcus samples. Analysis of the strength of correlation of each of the environmental variables highlighted wind direction, PAR, air temperature, tide, and rain as the strongest correlates. Wind direction has the strongest correlation at 47%, but all of these variables are strong predictors of Enterococcus with simple linear regression p-values less than 0.01.

FIG. 9 shows time-series comparisons of wind direction, tidal range, and inverse PA, verses maximum daily Enterococcus concentrations in the beach water. For wind direction, PAR, and air temperature, 24-hour average conditions were demonstrated to be the strongest predictors of Enterococcus based on simple linear regression p-values. Tide, rain, and wind speed had strong correlations up to 72 hours prior to sample times.

It was surprising that shorter term PAR, such as 3 or 6-hour averages, did not turn out to be stronger than the 24-hour average, given that the effect of solar radiation on FIB can deactivate bacteria in seawater. This would lead one to conclude that the strongest correlation between PAR and bacteria would be in the 3-hour timeframe. However, the data suggests that the natural beach environment and the turbidity levels in present in the water may allow bacteria to survive somewhat longer under sunlight. According to the data, prolonged strong sunlight for a full 24-hour period is needed for the most significant correlation with bacteria levels. That being said, the light level at the exact time of sampling was also evaluated and did show evidence of predictive capability, with a simple linear regression p-value of 0.04.

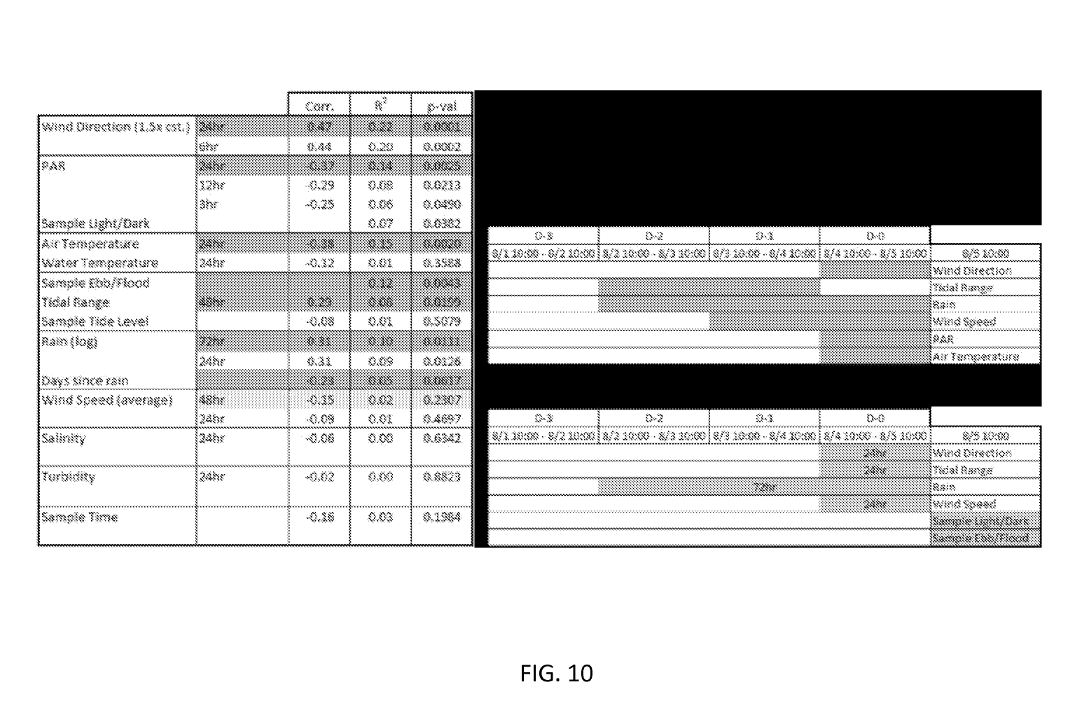

FIG. 10, left, shows a summary of statistical correlations between environmental variables and the beach samples. Strong correlations are highlighted in blue. A depiction of time periods with strongest correlations to Enterococcus are shown in the top right, and a depiction of time periods with strongest regression characteristics are shown on the bottom-right.

Air temperature was found to be a much stronger predictor of Enterococcus than water temperature, which was opposite the findings in Example 1, however this data could be a result of buoy placement. It also may be the case that the correlation between air temperature and Enterococcus is an indirect correlation through wind direction.

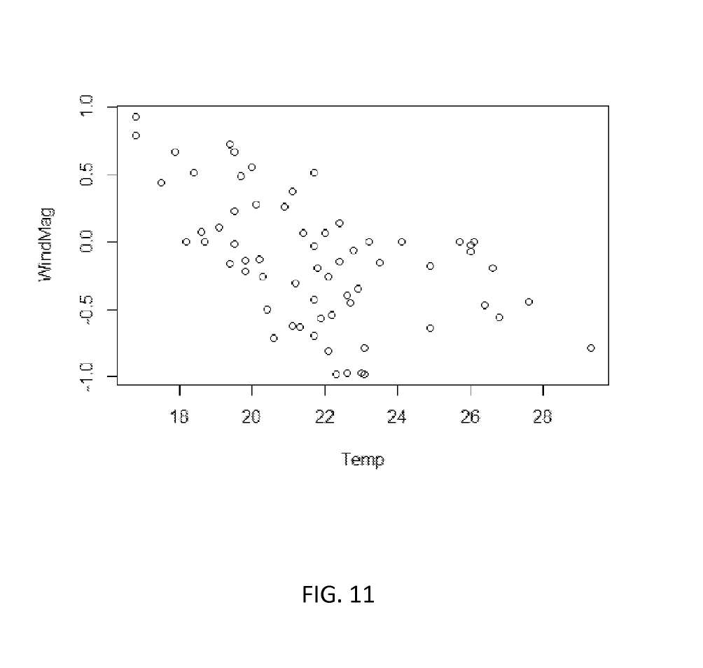

FIG. 11 shows a plot of wind direction verses air temperature. There is a strong visible correlation (correlation coefficient=52%) showing that air temperature tends to be warmer when the wind is blowing off of the beach and that wind direction was a very strong predictor of air temperature (regression p-value=1.1×10⁻⁵).

Tide conditions showed strong correlation with Enterococcus in two time dimensions. The strongest correlation was demonstrated by maximum tidal range in the period 24 to 72 hours prior to sampling. This suggests a time lag between conducive conditions and the presence of bacteria. This observed time lag suggests that high tides reinvigorate dormant bacteria in the beach and subsequent saturated conditions allow the bacteria to grow and enter the surf over a period of 24 hours or more. A second noteworthy time dimension was tidal phase at the time of sampling, which also showed a significant correlation with bacteria levels. The data showed strong evidence that bacteria levels during ebb tides were higher than bacteria levels during flood tides (one-sided t-test, p-value=0.0021) by at least 47 CFU (95% confidence interval). There are two logical explanations for this. It could support the hypothesis that the upper beach sands are a source of the bacteria, as the ebb tide could be drawing bacteria off the beach. Or, it could be explained as a result of high tides washing farther up into sewer drains and drawing dormant bacteria out of the drains.

Rain also showed evidence of being a strong predictor of bacteria levels on multiple dimensions. The strongest correlation was demonstrated by total rain for the previous 72 hours, but previous 24 and 48-hour rain totals were also significant with simple linear regression p-values less than 0.02 and sometimes performed better in the regression modeling. Days since the last rain (>0.1″) also showed some evidence of predictive capability with a p-value of 0.06. This study evaluated days since rain, 24-hour rain, and 72-hour rain in the subsequent regression modeling.

Wind speed showed little visible correlation and demonstrated no strength as an individual predictive variable, but turned out to be a strong predictive variable in multiple regression models. It is plausible that this reflects the value-added interaction effect of wind speed with wind direction. When onshore wind was combined with optimal wind speeds, the effect on water quality was probably accentuated.

Salinity and turbidity were also analyzed for correlation, but neither showed any significance. Salinity was inversely correlated with bacteria mortality and has been shown by other studies to have a negative relationship with Enterococci in the environment, but it seems that the salinity range (29-31 PSU) was not large enough at the beach sample site to be relevant. It was surprising that turbidity did not show more correlation, however, turbidity may be a redundant measure if wind and rain are included, since turbidity is primarily driven by these two variables.

Based on this analysis of individual variable correlations, the following variables were chosen for further analysis in regression models: 24-hour wind direction, 6-hour wind direction, 24-hour PAR, light level at time of sampling, 24-hour air temperature, 24-hour tidal range, 48-hour tidal range, tide phase at time of sampling, 24-hour total rain, 72-hour total rain, days since rain, 24-hour wind speed, and 48-hour wind speed. Through backward elimination, two best-fit models were developed:

Linear: ln(E)=3.0−0.55TP−0.71L+0.24T24+0.078ln(R24+10−4)+0.12T24M24

Logistic: ln(p/1−p)=3.96−2.6TP+0.21ln(R72+10−4)−0.40W24+0.22T24M24

Where:
E=modeled Enterococcus counts (CFU/100 mL)
p=modeled probability of exceeding swimming threshold
TP=tide phase at time of sampling (Flood=1, Ebb=0)
T=maximum tidal range (ft)
L=light level at time of sampling (Light=1, Dark=0)
R=total rainfall (in)
D=days since last rain greater than 0.1″
W=average wind speed (mph)
M=average wind magnitude or direction (vector −1 to 1)
P=average PAR (uE)
A=average air temperature (° C.)

Table 4 and Table 5 summarize the model development process with corresponding goodness of fit values for each step.

TABLE 4

| Variables | R² | F-statistic | p-value |
|---|---|---|---|
| M₂₄, P₂₄, A₂₄, T_{P}, In(R₇₂ + 10⁻⁴) , In(R₂₄ + 10⁻⁴), T₄₈, T₂₄, W₄₈, W₂₄, L, In(D + 1) | 0.471 | 3.64 on 12 + 49 d.f. | 0.0006 |
| M₂₄, P₂₄, A₂₄, T_{P}, In(R₇₂ + 10⁻⁴), In(R₂₄ + 10⁻⁴), T₄₈, T₂₄, W₄₈, L, In(D + 1) | 0.468 | 4.00 on 11 + 50 d.f. | 0.0003 |
| M₂₄, P₂₄, A₂₄, T_{P}, In(R₇₂ + 10⁻⁴), In(R₂₄ + 10⁻⁴), T₄₈, T₂₄, W₄₈, L | 0.466 | 4.46 on 10 + 51 d.f. | 0.0002 |
| M₂₄, P₂₄, T_{P}, In(R₇₂ + 10⁻⁴), In(R₂₄ + 10⁻⁴), T₄₈, T₂₄, W₄₈, L | 0.463 | 4.98 on 9 + 52 d.f. | 7.7 × 10⁻⁵ |
| M₂₄, T_{P}, In(R₇₂ + 10⁻⁴), In(R₂₄ + 10⁻⁴), T₄₈, T₂₄, W₄₈, L | 0.459 | 5.61 on 8 + 53 d.f. | 3.7 × 10⁻⁵ |
| M₂₄, T_{P}, In(R₇₂ + 10⁻⁴), In(R₂₄ + 10⁻⁴), T₂₄, W₄₈, L | 0.447 | 6.24 on 7 + 54 d.f. | 2.2 × 10⁻⁵ |
| M₂₄, T_{P}, In(R₂₄ + 10⁻⁴), T₂₄, W₄₈, L | 0.441 | 7.22 on 6 + 55 d.f. | 1.0 × 10⁻⁵ |
| M₂₄, T_{P}, In(R₂₄ + 10⁻⁴), T₂₄, L | 0.522 | 12.9 on 5 + 59 d.f. | 1.8 × 10⁻⁸ |
| M₂₄, T_{P}, In(R₂₄ + 10⁻⁴), T₂₄, L, T₂₄M₂₄ | 0.524 | 10.6 on 6 + 58 d.f. | 6.0 × 10⁻⁸ |
| T_{P}, In(R₂₄ + 10⁻⁴), T₂₄, L, T₂₄M₂₄ | 0.524 | 13.0 on 5 + 59 d.f. | 1.6 × 10⁻⁸ |
| Also evaluated M₆ and the following logical model - all worse | | | |
| M₂₄, T₂₄, T₂₄M₂₄, T_{P}, In(R₇₂ + 10⁻⁴) | 0.398 | 7.9 on 5 + 60 d.f. | 8.8 × 10⁻⁶ |

TABLE 5

| Variables | Res. deviance | AIC |
|---|---|---|
| M₂₄, P₂₄, A₂₄, T_{P}, In(R₇₂ + 10⁻⁴), In(R₂₄ + 10⁻⁴), T₄₈, T₂₄, W₄₈, W₂₄, L, D, T₂₄M₂₄ | 35.78 on 48 d.f. | 63.8 |
| M₂₄, P₂₄, A₂₄, T_{P}, In(R₇₂ + 10⁻⁴), In(R₂₄ + 10⁻⁴), T₄₈, T₂₄, W₄₈, W₂₄, L, T₂₄M₂₄ | 35.85 on 49 d.f. | 61.9 |
| M₂₄, P₂₄, A₂₄, T_{P}, In(R₇₂ + 10⁻⁴), T₄₈, T₂₄, W₄₈, W₂₄, L, T₂₄M₂₄ | 36.42 on 50 d.f. | 60.4 |
| M₂₄, P₂₄, A₂₄, T_{P}, In(R₇₂ + 10⁻⁴), T₄₈, T₂₄, W₂₄, L, T₂₄M₂₄ | 37.35 on 52 d.f. | 59.3 |
| M₂₄, P₂₄, T_{P}, In(R₇₂ + 10⁻⁴), T₄₈, T₂₄, W₂₄, L, T₂₄M₂₄ | 38.01 on 53 d.f. | 58.0 |
| M₂₄, P₂₄, T_{P}, In(R₇₂ + 10⁻⁴), T₄₈, T₂₄, W₂₄, T₂₄M₂₄ | 38.54 on 54 d.f. | 56.5 |
| M₂₄, P₂₄, T_{P}, In(R₇₂ + 10⁻⁴), T₂₄, W₂₄, T₂₄M₂₄ | 38.94 on 55 d.f. | 54.9 |
| M₂₄, P₂₄, T_{P}, In(R₇₂ + 10⁻⁴), W₂₄, T₂₄M₂₄ | 39.47 on 56 d.f. | 53.5 |
| M₂₄, T_{P}, In(R₇₂ + 10⁻⁴), W₂₄, T₂₄M₂₄ (large negative coefficient on M24) | 40.82 on 57 d.f. | 52.8 |
| T_{P}, In(R₇₂ + 10⁻⁴), W₂₄, T₂₄M₂₄ | 43.52 on 58 d.f. | 53.5 |
| Also evaluated removing T₂₄M₂₄ or W₂₄ and following logical model - all worse | | |
| M₂₄, T₂₄, T₂₄M₂₄, T_{P}, In(R₇₂ + 10⁻⁴) | 50.05 on 60 d.f. | 62.1 |

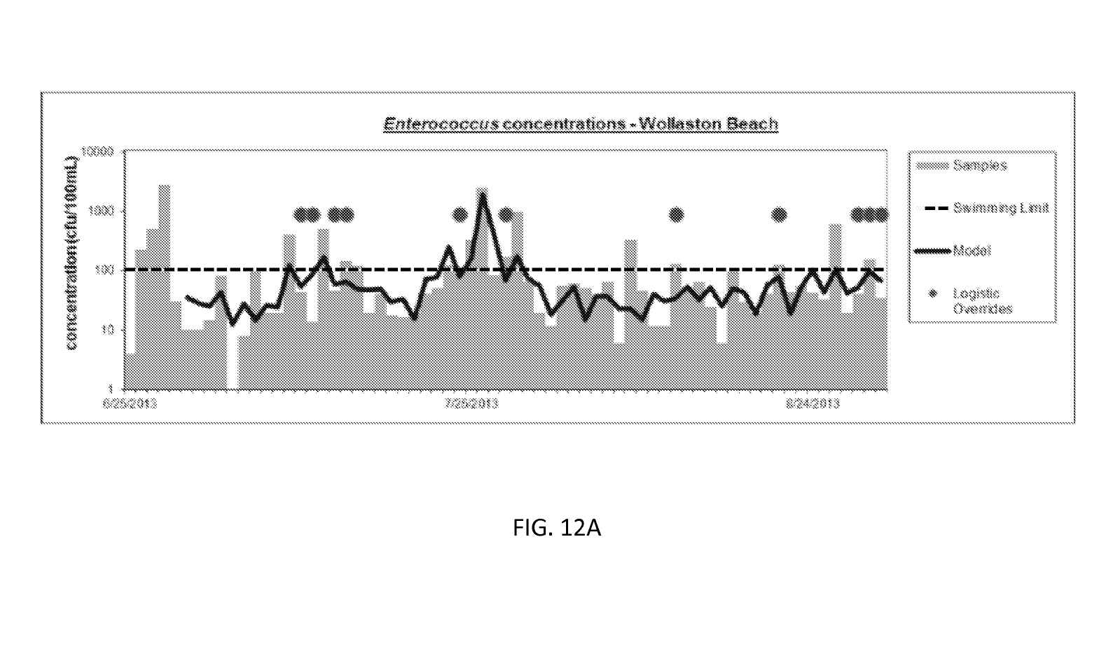

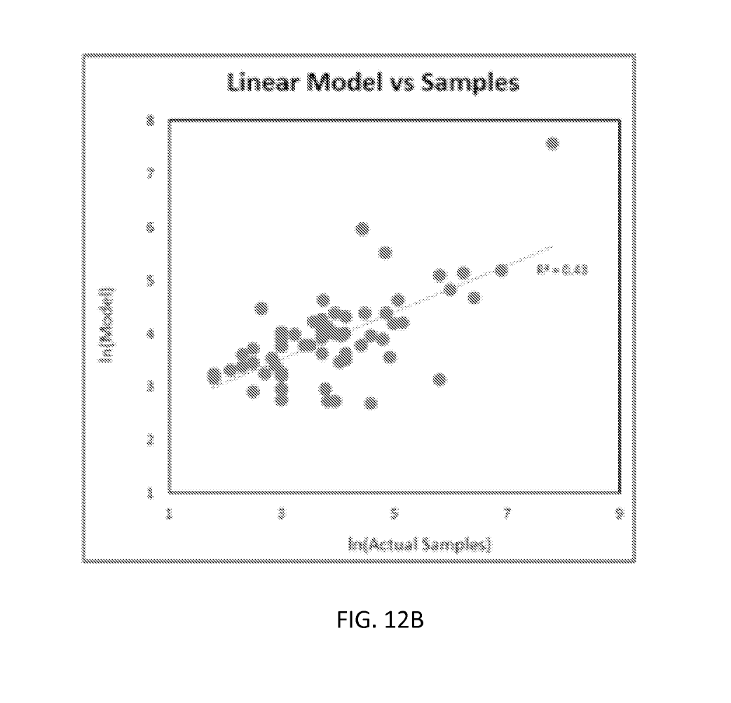

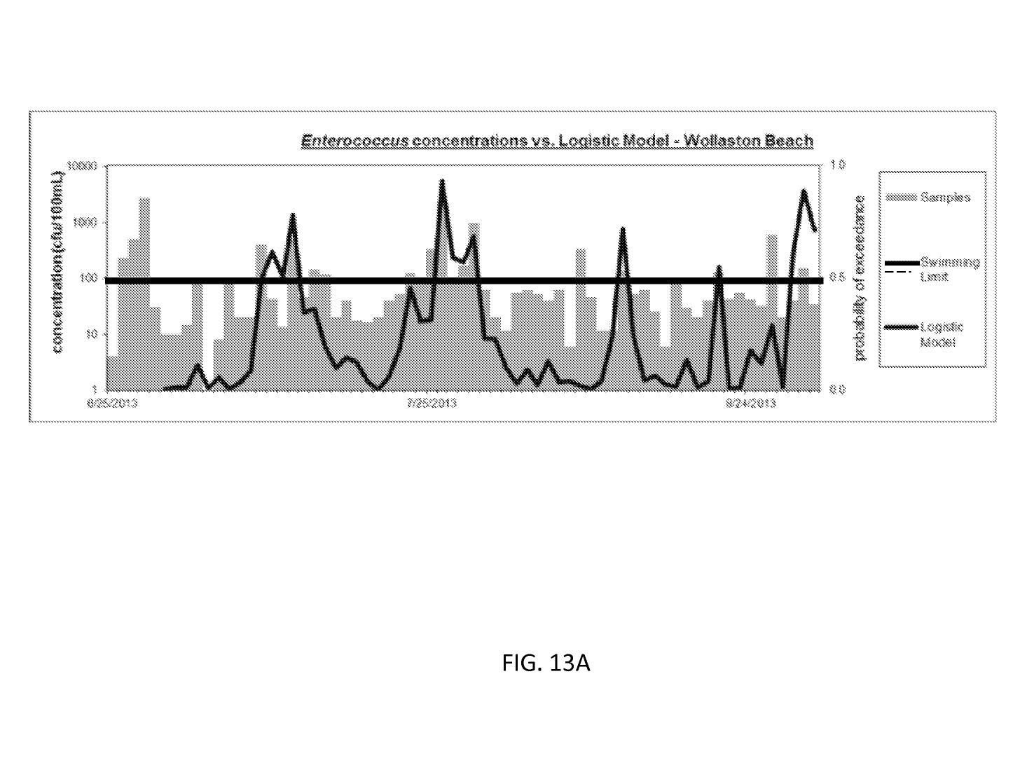

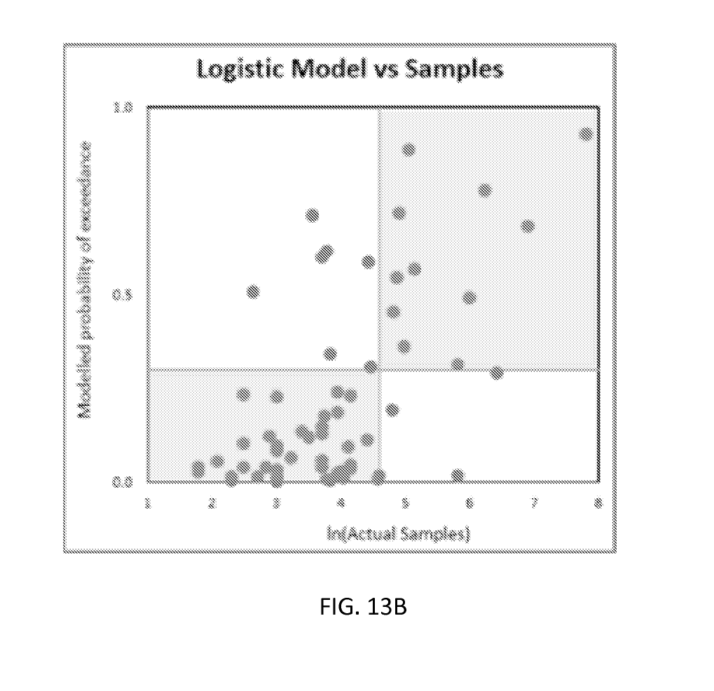

Once models were developed, it was possible to study the predictive strength of the models and the value of real-time sensors and real-time predictions. The beach models did not turn out as strong at predicting bacterial exceedances as the river models, possibly because conditions are less unidirectional and more directionally chaotic in a bay than in a river. Linear model performance is shown in FIGS. 12A and 12B. The daily data, linear model had a true-positive rate (sensitivity) of 50% and a true-negative rate (specificity) of 98%. In other words, when run once per day at sampling time, it correctly predicted 50% of actual bacterial exceedances and 98% of actual good water quality days. The logistical model performance is shown in FIGS. 13A and 13B. The logistical model produced better results, when evaluated against a 0.3 probability threshold, with 79% true-positive rate and 85% true-negative rate. The probability threshold is arbitrary, and can be adjusted to produce the best fit. These numbers may be considered low, but in comparison with the predictive results of using the previous day samples, they are a significant improvement. Using previous day samples to predict current bacteria levels resulted in a sensitivity of only 21%. A model which evaluated previous day bacteria together with 24-hour rain also resulted in the same sensitivity of 21%.

In at least some embodiments, local and distant data sources may be compared using a derived regression model by analyzing relative sensitivity and selectivity. In accordance with one or more embodiments, local data is more accurate by at least about 5%.

According to the invention, hourly, instead of daily, predictions can be made using sensors and a model, changing how a user will use a recreational area. The invention may increase local business derived from swimmers who may have been unable to swim for an entire day when some of the day actually had acceptable water quality for swimming. Similarly, if the daily prediction misses a temporary spike, it may not protect human health whereas the hourly model predictions may decrease risk to human health. The public's increased awareness of acceptable swimming times may increase safe recreation and decrease human health risk. As such, an app or other information delivery system may be widely used and could become a common component of smart phones for planning beach use. The information delivery system may publish predictions more than once per day, such as hourly, similar to weather prediction apps that publish more often than once per day.

Having now described some illustrative embodiments, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

It is to be appreciated that embodiments of the devices, systems and methods discussed herein are not limited in application to the details of construction and the arrangement of components set forth in the following description or illustrated in the accompanying drawings. The devices, systems and methods are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. An autonomous environmental data collection station configured to provide real-time localized data associated with a body of water, the station comprising:
a buoyant base;
a plurality of sensors affixed to the base and suspended at a constant depth in the body of water;
a data logger in communication with the plurality of sensors, the data logger having cellular telemetry capability;
at least one battery constructed and arranged to power the plurality of sensors and the data logger; and
at least one solar panel constructed and arranged to charge the at least one battery.

2. The station of claim 1, wherein the plurality of sensors collect data on at least one of wind speed, photosynthetically active radiation, air temperature, humidity, water temperature, barometric pressure, salinity, conductivity, and turbidity.

3. The station of claim 2, wherein the plurality of sensors provides collected data to the data logger at least every two hours.

4. The station of claim 3, wherein the plurality of sensors provides collected data to the data logger about every 10 minutes.

5. The station of claim 4, where the data logger is configured to communicate with a web-based database through cell-phone based telemetry.

6. The station of claim 5, wherein the data logger exports data to the web-based database about every two hours.

7. A water quality monitoring system, comprising:
an autonomous environmental data collection station in communication with a body of water and configured to provide real-time localized data on at least one parameter associated with the body of water;
a processor configured to receive the real-time localized data collected by the data collection station, manipulate the real-time localized data based on a predictive water quality model to determine a predictive bacteria level of the body of water, compare the predictive bacteria level to a threshold value, and output a safety recommendation based on the comparison; and
a display in communication with the processor and configured to display the safety recommendation;
wherein the system has an operative sensitivity of at least about 80% or a specificity of at least about 85%.

8. The water quality monitoring system of claim 7, wherein the data collection station is disposed on land proximate the body of water.

9. The water quality monitoring system of claim 7, wherein the data collection station is disposed on a float and positioned in the body of water.

10. The water quality monitoring system of claim 7, wherein the processor is further configured to receive non-localized data, and input the non-localized data into the predictive water quality model.

11. The system of claim 10, wherein the additional data comprises non-localized weather data and water flow data.

12. The system of claim 11, wherein the processor is further configured to manipulate the predictive bacteria level to a binary probability between 0 and 1.

13. The system of claim 12, wherein the binary probability corresponds to the predicted bacteria level of the body of water.

14. The system of claim 13, wherein the predictive model predicts e. coli bacteria levels in the body of water.

15. The system of claim 13, wherein the predictive model predicts Enterococcus bacteria levels in the body of water.

16. The system of claim 13, wherein the predictive model is an algorithmic model.

17. The system of claim 17, wherein the algorithmic model is self-updating.

18. A method of generating a model to predict bacteria levels in a body of water, the method comprising:
disposing an autonomous environmental data collection station at the body of water, the autonomous environmental data collection station comprising a plurality of sensors configured to provide real-time data on a plurality of environmental parameters related to the body of water;
exporting the real-time data from the plurality of sensors to an offsite database in regular intervals;
simultaneously collecting water samples from the body of water and measuring the bacteria level in the body of water;
sourcing data on additional environmental parameters from non-localized data collection stations;
analyzing each environmental parameter to determine its predictiveness of bacteria level;
selecting a plurality of analyzed environmental parameters based on their predictiveness of bacteria level; and
using the selected environmental parameters to derive a predictive water quality model.

19. The method of claim 18, wherein each of the plurality of environmental parameters are analyzed with linear regression.

20. The method of claim 19, wherein the predictive water quality model is used to provide safety recommendations concerning the body of water.
